SEQUENCE LISTING (26 sequences)

SEQ ID NO: 1 - DNA, 22 bp, Artificial Sequence
  Synthetic Primer
tcttcacatatgagcaccaacc

SEQ ID NO: 2 - DNA, 21 bp, Artificial Sequence
  Synthetic Primer
cggaacaagaggaagtgagtg

SEQ ID NO: 3 - DNA, 22 bp, Artificial Sequence
  Synthetic Primer
ttgtacaatcccagtggaatcc

SEQ ID NO: 4 - DNA, 22 bp, Artificial Sequence
  Synthetic Primer
aatagggcatgagggtgtttga

SEQ ID NO: 5 - DNA, 20 bp, Artificial Sequence
  Synthetic Primer
cattcaacctggaatctggg

SEQ ID NO: 6 - DNA, 19 bp, Artificial Sequence
  Synthetic Primer
tccctggaaaggctacacc

SEQ ID NO: 7 - DNA, 23 bp, Artificial Sequence
  Synthetic Primer
tgagaggtcagttacagaagacc

SEQ ID NO: 8 - DNA, 20 bp, Artificial Sequence
  Synthetic Primer
gatcctcctccaaatcccat

SEQ ID NO: 9 - DNA, 18 bp, Artificial Sequence
  Synthetic Primer
acctgccagagttgtggc

SEQ ID NO: 10 - DNA, 22 bp, Artificial Sequence
  Synthetic Primer
aagagtttcatttctcccatcc

SEQ ID NO: 11 - DNA, 20 bp, Artificial Sequence
  Synthetic Primer
ttacttctttggatcatgtc

SEQ ID NO: 12 - DNA, 20 bp, Artificial Sequence
  Synthetic Primer
gtccctcctcataatttctg

SEQ ID NO: 13 - DNA, 20 bp, Artificial Sequence
  Synthetic Primer
tgctctaatctacccgggtc

SEQ ID NO: 14 - DNA, 18 bp, Artificial Sequence
  Synthetic Primer
ccaccccactcccttctg

SEQ ID NO: 15 - DNA, 23 bp, Artificial Sequence
  Synthetic Primer
tgagaggtcagttacagaagacc

SEQ ID NO: 16 - DNA, 20 bp, Artificial Sequence
  Synthetic Primer
gatcctcctccaaatcccat

SEQ ID NO: 17 - DNA, 22 bp, Artificial Sequence
  Synthetic Primer
tcatcaccatcataccaagatg

SEQ ID NO: 18 - DNA, 21 bp, Artificial Sequence
  Synthetic Primer
aattctgccaagaagagagcc

SEQ ID NO: 19 - DNA, 20 bp, Artificial Sequence
  Synthetic Primer
ctttgctccccattaagctg

SEQ ID NO: 20 - DNA, 22 bp, Artificial Sequence
  Synthetic Primer
tctagcaaatgtctgagatgcc

SEQ ID NO: 21 - DNA, 21 bp, Artificial Sequence
  Synthetic Primer
ccatctcatccagaaacatcc

SEQ ID NO: 22 - DNA, 20 bp, Artificial Sequence
  Synthetic Primer
aggatgctgaggtgttaggc

SEQ ID NO: 23 - DNA, 20 bp, Artificial Sequence
  Synthetic Primer
cattcaacctggaatctggg

SEQ ID NO: 24 - DNA, 19 bp, Artificial Sequence
  Synthetic Primer
tccctggaaaggctacacc

SEQ ID NO: 25 - DNA, 22 bp, Artificial Sequence
  Synthetic Primer
catgagattcagcgattcatcg

SEQ ID NO: 26 - DNA, 22 bp, Artificial Sequence
  Synthetic Primer
tgctctggcttaaacttctccg

(19) United States
(12) Patent Application Publication
(10) Pub. No.: US 2004/0106111 A1
(43) Pub. Date: Jun. 3, 2004

(54) Assay

(76) Inventors: Christopher Simon Haley, Edinburgh (GB); Alan Langskill Archibald, Edinburgh (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/182,952

(22) PCT Filed: Feb. 5, 2001
(86) PCT No.: PCT/GB01/00448

(30) Foreign Application Priority Data
Feb. 4, 2000 (GB) 0002451.3

(51) Int. Cl. C12Q 1/68

(52) U.S. Cl. 435/6

(57) ABSTRACT

The present invention provides a method for determining the predisposition of pigs to boar taint. Boar taint is a strong unpleasant odour given off upon heating or cooking of meat from uncastrated male pigs. Boar taint is associated with elevated levels of skatole, indole and androstenone. There are significant economic losses attributable to current methods of preventing or producing the effect of boar taint. Thus, the identification of animals of the desired genotype allows for the selection against animals with a genetic predisposition to boar taint, this being an attractive, cost effective and humane solution to the boar taint problem. The present invention thus identifies QTL for boar taint and its component traits. In particular said traits are shown to be particularly located on chromosome 6 and 14, and further an important candidate gene mapping to chromosome 14 is also shown.

The present invention relates to genetic markers for pigs exhibiting desirable flavour properties. In particular, the present invention provides an assay to screen pigs for boar taint and its associated flavours. Generally pigs having low boar taint levels will be positively selected, but it is also possible to identify animals having unacceptably high boar taint levels.

Boar taint is a strong perspiration-like, urine-like unpleasant odour given off upon heating or cooking of meat from some entire (uncastrated) male pigs. The off-odours and off-tastes, commonly known as “boar taint”, are objectionable to consumers. In the United States carcasses tainted by boar odour are either condemned or subject to restricted use by United States Department of Agriculture meat inspectors EU law (Council Directive 91/497/EEC, which has been implemented in Britain through the Fresh Meat (Hygiene and Inspection) Regulations 1992) states that animals over 80 kg carcase weight, excluding the head, should be screened for boar taint, but no method is specified.

The most effective method, to date, for preventing “boar taint” is to castrate (remove the testes of) young male pigs. Castration of young male pigs is widely practised in pig production systems in North America and Europe. However, as outlined below, there are production advantages of using entire male pigs. Entire male pigs are used extensively in pig production in the United Kingdom and also in Denmark, Australia and parts of Spain. Other measures taken to reduce the risk of boar taint include slaughtering entire male pigs at an earlier age than castrated males.

Pig production systems that involve castration of young male pigs suffer economic losses and other disadvantages. These economic losses are attributable to lost opportunities to access the superior performance, especially feed conversion, of intact males and the inferior nature of carcasses from castrates (barrows) (for example: Allen, P., Riordan, P. B., Hanrahan, T. J. and Joseph, R. L. 1981. Production and quality of boar and castrate bacon. Irish J. Sci. Technol. 5, 93-104; Wood, J. D. and Riley, J. E. 1982. Comparison of boars and castrates for bacon production. 1. Growth data, and carcass and joint composition. Animal Production 35, 55-63; Ellis, M., Smith, W. C., Clark, J. B. K. and Innes, N. 1983. A comparison of boars, gilts and castrates for bacon manufacture. 1. on farm performance, carcass and meat quality characteristics and weight loss in the preparation of sides for curing. Animal Production 37, 1-9). If the problem of boar taint were overcome, raising boars rather than castrates would have considerable economic advantages. Although boars and castrates gain weight at equivalent rates, boars produce carcasses containing 20-30% less fat. Boars also utilise feed more efficiently than castrates (10% less feed consumed per unit of body weight). Since feed represents the major cost in pig production, raising boars for pork would have significant economic advantages.

Castration not only produces animals with inferior carcass characteristics and a less efficient feed conversion, but is also bad for the pig's welfare. Adverse animal welfare considerations include the pain associated with castration, the loss of ‘normal’ behaviour and the risk of infection.

In conclusion, there is a need for methods to prevent or determine predisposition to boar taint, that do not require castration of young pigs.

### Boar Taint

Boar taint is associated with elevated levels of androstenone (5-androst-16-en-3-one), indole and skatole (3-methyl-1H-indole) (Patterson, 1968; Bonneau, 1982; see also Claus et al. 1994. Physiological aspects of androstenone and skatole formation in the boar—a review with experimental data. Meat Science 38, 289-305).

Androstenone gives a urine or perspiration-like odour, whilst indole and skatole give a camphor-like odour. Levels of androstenone and skatole are each increased in non-castrated boars, although the reason for increased skatole levels has not been established. Additionally the formation of androstenone and skatole appears to be independent although the degradation of these compounds is currently believed to follow similar pathways and may each involve cytochrome P450s. There remains debate concerning the relative importance of androstenone and skatole in contributing to boar taint, and in certain studies emphasis has been placed onto androstenone (see WO 98/41861 and WO 99/18192).

Methods that address the variation in levels of both compounds would be particularly useful for breeding male slaughter pigs.

The 16-androstene steroids, such as 5-androst-16-en-3-one (androstenone), are produced in the Leydig cells of the testis and passed into the bloodstream (Bonneau, 1982). Due to their hydrophobic nature, 16-androstene steroids are subsequently absorbed by fatty tissues.

Skatole (3-methyl-indole) is produced by the breakdown of tryptophan by bacteria in the hind gut of pigs and other animals (see Moss et al., “Boar taint: the role of skatole”, Meat Focus International, October 1992; and Babol et al., “Boar taint in entire male pigs”, EAAP Publication No 92). Skatole is absorbed into the bloodstream and deposited in fatty tissues.

Methods for the identification and production of swine with reduced boar taint are described In WO 99/18192. The method of WO 99/18192 is concerned with androstenone production and in particular the predicted impact of specific natural or experimentally-induced mutations or polymorphisms in the porcine CYP17 gene which encodes cytochrome P450c17. Cytochrome P450c17 is required for production of androstenone. A method for determining predisposition to boar taint is disclosed in WO 98/41861. The method of WO 98/41861 is concerned with assaying for the presence of a low molecular weight isoform of cytochrome b5. Cytochrome b5 is involved with cytochrome P450c17 in the synthesis of androstenone. Although data relating levels of cytochrome b5 to levels of androstenone are presented no evidence of a genetic component of the differences is presented.

Neither the methods of WO 99/18192 nor WO 98/41861 address the contribution of skatole or indole. Skatole is critical to consideration of ‘boar taint’. While about 25% of consumers are not able to smell androstenone (Claus, 1978. Wien. Tierartztl Mschr 65, 381) skatole is detected by all consumers. Moreover, as skatole formation is not limited to the boar, an understanding of skatole production and clearance may be valuable in other meat species.

Previous research has suggested that part of the variation in boar taint or its component traits is under genetic control.

Willeke et al., (Willeke et al., 1987. Selection for high and low level of 5-androst-16-en-3-one in boars. I. Direct and correlated response of endocrinological traits. Journal of Animal Breeding and Genetics 104, 64-73) and Sellier and Bonneau (Sellier and Bonneau, 1988. Genetic relationships between fat androstenone level in males and development of male and female genital tracts in pigs. Journal of Animal Breeding and Genetics 105, 11-20) have shown that selection (i.e. selective breeding) on fat androstenone level in boars can be effective. Keller et al. (Keller et al., 1997. Influencing the androstenone concentration of entire male pigs by mating AI boars with known fat androstenone level. EAAP Working Group “Production and utilisation of meat from entire male pigs”, Stockholm, Sweden, 1-3 Oct. 1997) confirmed that there is a genetic component to androstenone levels. Lundström and co-workers concluded from a study of skatole levels in pig selection lines that there is a genetic effect on skatole deposition which may be due to a recessive allele of a major gene (Lundtröm et al., 1994. Skatole levels in pigs selected for high lean tissue growth rate on different dietary protein levels. Livestock Production Science 38, 125-132). Fouilloux and colleagues (Fouilloux et al., 1997. Support for single major genes influencing fat androstenone level and development of bulbo-urethral glands in young boars. Genetic Selection Evolution 29, 357-366; Le Roy et al., 1997. Evidence for single major genes influencing fat androstenone level and development of bulbo-urethral glands in young boars. EAAP Working Group “Production and utilisation of meat from entire male pigs”, Stockholm, Sweden, 1-3 Oct. 1997) concluded from their data that there is a single major gene influencing androstenone levels in fat. In their model the allele for ‘low androstenone levels’ is dominant with respect to the allele for ‘high androstenone levels’. They found no evidence for linkage between the major genes for androstenone levels and bulbo-urethral gland development and the swine leukocyte antigen loci (SLA). However, Bidanel et al. (Bidanel et al., 1997. Chromosome 7 mapping of a quantitative trait locus for fat androstenone level in Meishan×Large White F2 entire male pigs. EAAP Working Group “Production and utilisation of meat from entire male pigs”, Stockholm, Sweden, 1-3 Oct. 1997) found evidence for an effect on androstenone levels of a gene or genes on chromosome 7, close to the SLA locus. The androstenone QTL described by Bidanel and colleagues maps to the interval SLA-S0102 that approximately corresponds to the TNFB-S0066 interval in our study.

### Genetic Selection

Selection against animals with a genetic predisposition to boar taint would be an attractive, cost-effective and humane solution to the problem of boar taint. The identification of animals of the desired genotype (genetic make up) requires some understanding of the nature of genetic variation and methods to detect it.

### The Genome and Genetic Variation

The genome of the pig consists of a set of 18 pairs of autosomes and the sex (X and Y) chromosomes found in most cells of the animal. Into these chromosomes is packed a DNA sequence of around 3 billion base pairs in length. This DNA sequence codes for the 50,000 to 100,000 genes that control the development of the pig and its appearance, performance and other characteristics. Slight variations in the DNA sequence between animals contribute to differences between animals within breeds and between breeds. The two copies of a gene carried by an animal on alternative members of a homologous chromosome pair may differ from each other in their exact DNA sequence. These alternative variants (or alleles) may or may not encode functionally different products, depending upon the exact nature of the change at the DNA level. Such variation found in a population is referred to as polymorphism and genes or loci displaying variation are said to be polymorphic.

An animal's phenotype is the result of complex actions of the genes inherited from its parents and environmental factors. Most traits of agricultural importance in pigs are influenced by variation at several or many different genes. Usually individual genes do not have a large enough effect on their own to produce observable qualitative differences between individuals. More commonly, variation in several or many genes combines to produce continuous or quantitative variation between animals in traits such as growth rate, fatness and predisposition to boar taint.

Genome mapping can be used to identify the location of genes that influence variation in quantitative traits. For example, if it can be demonstrated that there are significant associations between the inheritance of a particular chromosomal region (or locus) and trait variation, that region must contain a gene or genes affecting the trait in question. The loci affecting quantitative traits are termed quantitative trait loci or QTLS.

The tools used to follow the inheritance in different chromosomal regions are genetic markers and these can be selected from the genome map to ensure coverage of the entire genome. Markers on the genetic map are used to identify a particular region of the genome and follow its inheritance and thus provide the tools to find genes affecting traits of interest.

The most commonly used markers are microsatellites, where the core of the marker is a tandemly-repeated sequence of two (usually) or a small number of nucleotides, where different alleles are distinguished by having different numbers of repeats. For microsatellites (and for many of the other possible marker types), the polymerase chain reaction (PCR) is used to amplify a small DNA sample and provides the first step in identifying alternative alleles (i.e. genotyping). Unique PCR primers are used to ensure that only alleles of the specific marker of interest are amplified from the DNA sample of an individual animal. The PCR products are then separated by electrophoresis and can be visualised, for example via use of radioactive or fluorescent labels. The use of PCR on DNA-based markers means that genotyping can be performed on very small samples of DNA, which can be relatively easily collected at any time. Hence animals can be genotyped as soon as they are born using DNA isolated from blood, ear notches or other material.

The genetic map can be built in a number of ways, however, the principle method is by linkage analysis. If two markers are close together on a chromosome, then the two alleles that are on the same gamete of an individual will tend to be inherited together. The closer together these two loci are, the more likely it is that they will not be separated by recombination and so will appear linked. Alleles at two loci far apart on the same chromosome or on different chromosomes will be inherited independently and so will produce a proportion of 0.5 recombinant and 0.5 non-recombinant gametes. Hence the frequency of recombinants (the recombination fraction) provides a measure of the distance between two loci. Maps showing distances between ordered loci can be built using recombination frequencies between pairs of loci or between multiple groups of loci.

Linkage maps of the porcine genome now contain substantial amounts of information and their status is constantly changing. Published linkage maps and linkage data are stored in the pig genome database (PiGBASE/ARKdb-pig: URL=

http://www.ri.bbsrc.ac.uk/pigmap/pig genome mapping.h tml.

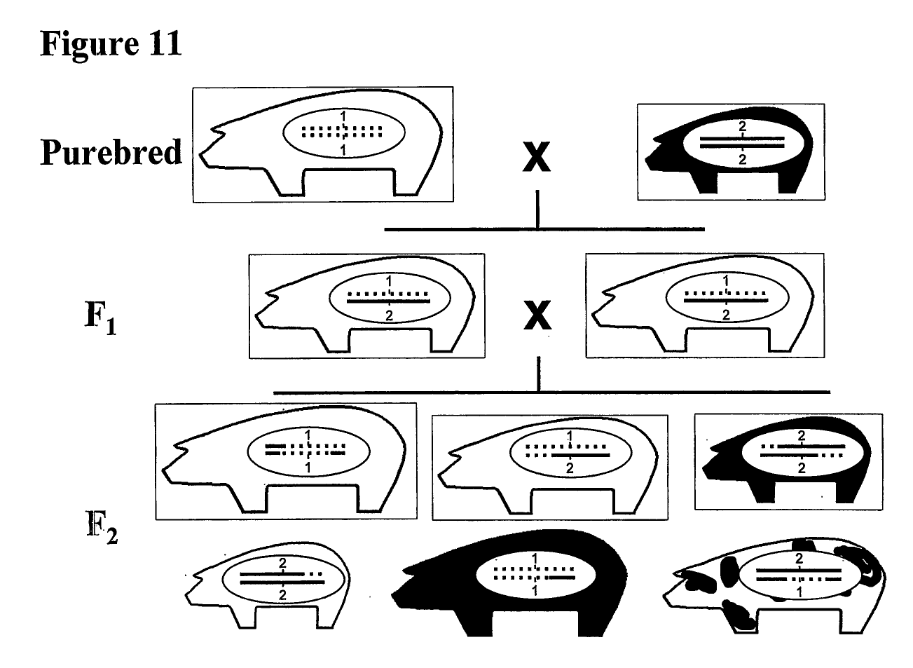

The basic principle of showing that a gene or a region of the genome is associated with variation is illustrated in FIG. 11. It consists of identifying a genetic marker and showing that its inheritance in a suitable pedigree is associated with variation in performance.

In a population such as that derived from the cross between two lines illustrated in FIG. 11, there may be an overall association between a particular marker allele and a particular allele at a quantitative trait locus (QTL). In other words, on average, across all individuals no matter which family they come from, there is a tendency for a particular marker allele to be associated with a particular QTL allele. Such an association is often referred to as linkage disequilibrium. Linkage disequilibrium between a QTL and a marker leads to an overall association between the marker allele and the quantitative trait. In a random mating population, recombination will lead to the gradual decay in linkage disequilibrium between loci, with the rate of decay related to the distance between the loci.

In the analysis of data, one can look for an overall association between a marker and a quantitative trait (an association study). In such an analysis one is making the assumption that the marker and the QTL are in linkage disequilibrium, perhaps because they are very close together (e.g. within the same candidate gene), or because the population is not long established. However, even if a marker and a QTL are very close together, there is no guarantee that linkage disequilibrium between them exists (except in special circumstances, such as a cross between inbred lines) and so a QTL may be missed if association analysis is performed alone. Linkage analysis is a more robust test, as it will detect both associations that vary between families and those that are consistent across the population. However, depending on the population structure, it may be more difficult to perform linkage analysis than association analysis. This is particularly because linkage analysis requires the data to be sampled in a designed manner from a population carefully structured into families, whereas association analysis can be performed on a random sample of individuals. Thus linkage analysis is not always carried out, even though it would be optimum to perform both types of analysis.

Genome studies often analyse several or many different markers when looking for an effect on the phenotype. Thus, a number of effects may be significant by chance if the standard 5% significance level is used. Hence, it is recommended practise to use a more stringent significance level such that the overall chance of finding a significant result amongst all the markers tested is no more than 5% (see Lander and Kruglyak, 1995, for a more detailed discussion of these points). This means that significance levels as high as 0.01-0.001% may be used in some studies. This in turn increases the sample size required for results to be significant at this level. The samples sizes required to be confident of detecting an effect depend on factors such as the magnitude of the influence on the trait, the type of population studied and the exact analysis to be performed. However, even in the most straightforward situation and with the most carefully designed studies, the minimum sample sizes are likely to be two hundred animals or more.

The full power of the map and markers is employed in performing a genome scan for loci affecting traits of interest. The strength of this approach is that it has the potential to detect any loci with a large effect on a studied trait, whether or not their existence is known in advance. To implement this approach markers which are spaced at intervals through the genome and which are polymorphic in the population being studied are selected from the map. The phenomenon of genetic linkage means that each marker can be used to follow the inheritance of a section of linked chromosome. Around 100-150 evenly spaced markers are needed to cover the whole genome and follow the inheritance of all sections. Thus maps of highly polymorphic markers are very valuable for this approach, as they allow selection of markers that provide this coverage and that are informative in the population of interest.

Thus the genome scan can both localise known genes of major effect and identify loci that were not known a priori. A significant amount of work is required to type sufficient animals for markers covering the entire genome. However, it is possible to design an experiment such that there is a high probability of detecting a gene of a defined effect on the phenotype wherever it is in the genome.

We have conducted such a genome scan for QTL contributing to variation in boar taint and its component traits.

We have identified QTL for boar taint and its component traits. Of most interest are QTL for boar taint traits located on chromosome 6 (in a region defined by the markers SW782, SW1057, S0121 and SW322) and on chromosome 14 (in a region defined by the markers SW857, SW2496, SW295, SW210, S0007, SW761 and SW1557). We have also identified further QTL with smaller effects for different components of boar taint on several other chromosomes. (e.g. 1, 2, 3, 4, 5, 8, 9, 10, 11, 13, 18 and X).

Thus, in one aspect, the present invention provides genetic markers for characteristics of boar taint, derived from:

i) SW782, SW1057, S0121, SW322 or regions of chromosome 6 spanning therebetween (preferably between positions 40 to 120 of chromosome 6); or

ii) SW857, SW2496, SW295, SW210, S0007, SW761, SW1557, SW2515, SWC27 or regions of chromosome 14 spanning therebetween (preferably between positions 10 to 70 of chromosome 14).

The specific markers referred to above detailed in the website

http://www.ri.bbsrc.ac.uk/pigmap/pigbase/pigbase.html

and specifically can be accessed via

http://www.ri.bbsrc.ac.uk/pigmap/pigbase/loclist.html

Brief details of these markers are also set out in the example.

In a further aspect, the present invention provides an assay to identify pigs with a genetic predisposition that reduces the incidence of boar taint, wherein said assay comprises:

a) obtaining a DNA sample from a test pig;

b) analysing the sample to determine the allelic variant(s) present at a genetic marker, wherein said markers are selected from:

i) SW782, SW1057, S0121, SW322, or regions of chromosome 6 spanning therebetween (preferably between positions 40 to 120 of chromosome 6); or

ii) SW857, SW2496, SW295, SW210, S0007, SW761, SW1557, SW2515, SWC27 or regions of chromosome 14 spanning therebetween (preferably between positions 10 to 70 of chromosome 14; and

c) using said marker results to select for animals of the preferred genotype.

In a yet further aspect, the present invention provides a method of identifying boars which have a genetic disposition to reduced boar taint, said method comprising:

a) obtaining a DNA sample from said boar;

b) assaying said boar for a sequence identical or complementary to the genetic markers identified above.

Although the study looked at the particular markers identified above, it is known to those skilled in the art that other genetic markers from within the QTL or the neighbouring portions of chromosome 6 or 14 (as appropriate) may be used instead, provided of course that the marker(s) selected are found to map within or close to the QTL for boar taint traits.

Thus, the present invention provides a method to identify pigs with a genetic predisposition that reduces the incidence of boar taint, wherein said method comprises:

a) obtaining DNA samples from a population of pigs;

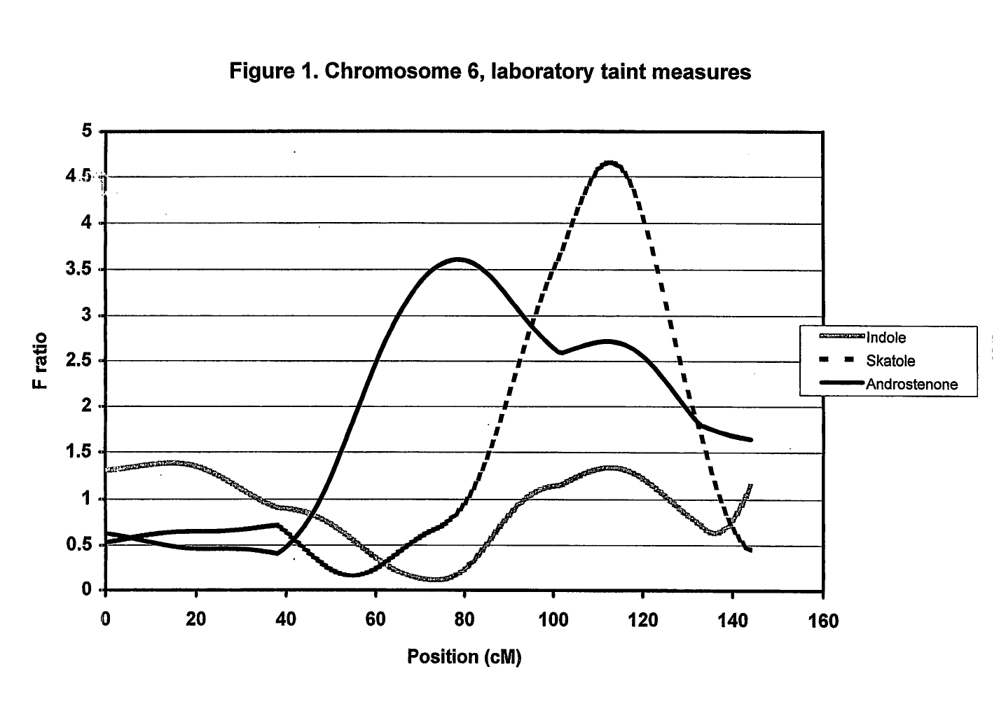

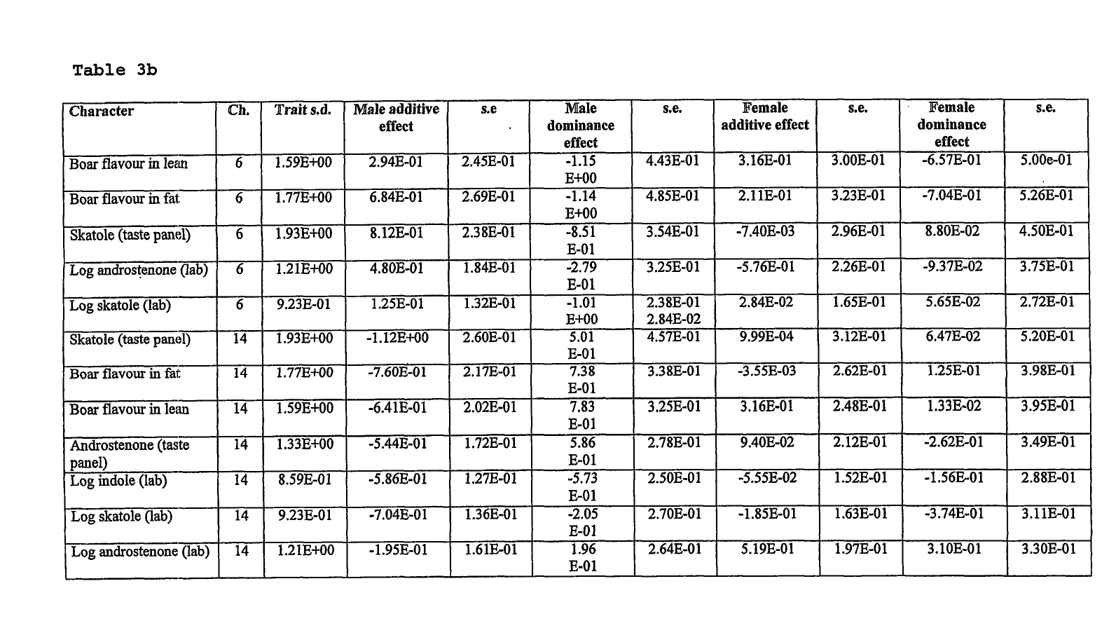

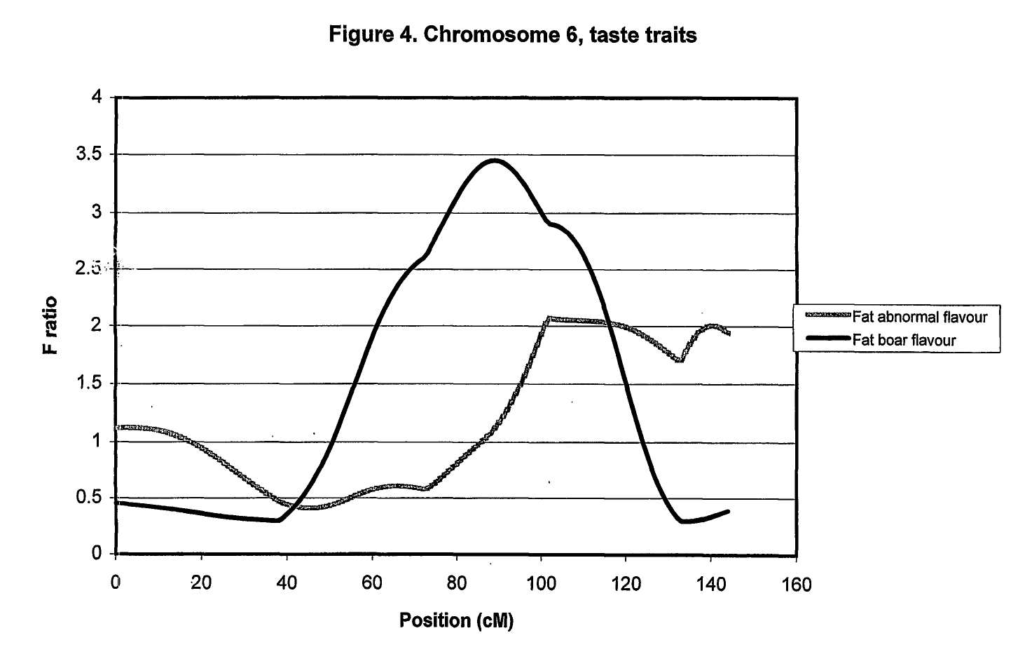

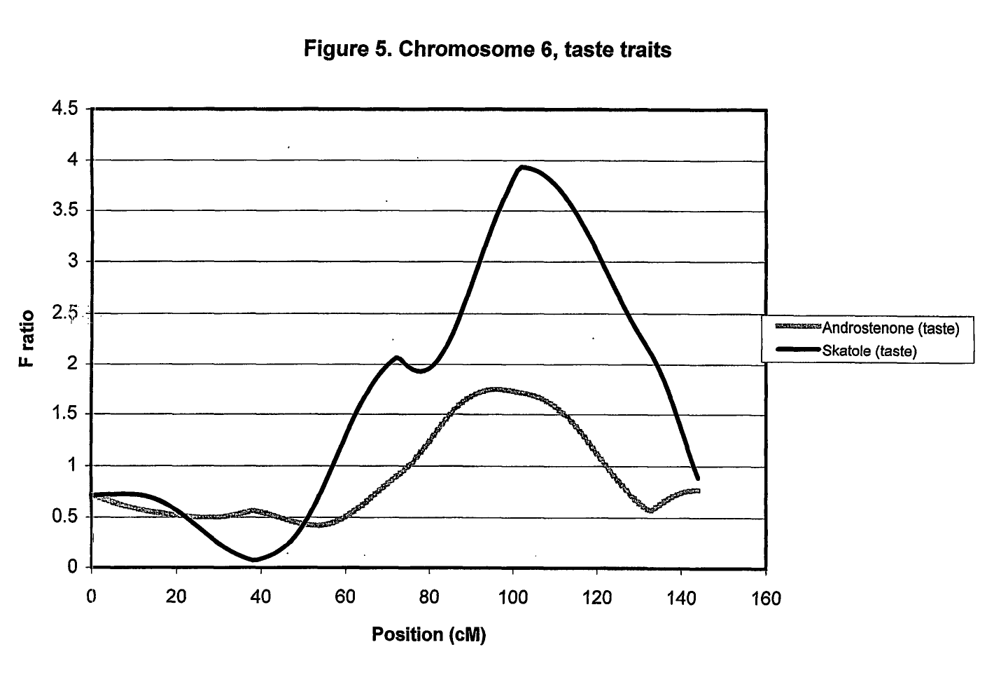

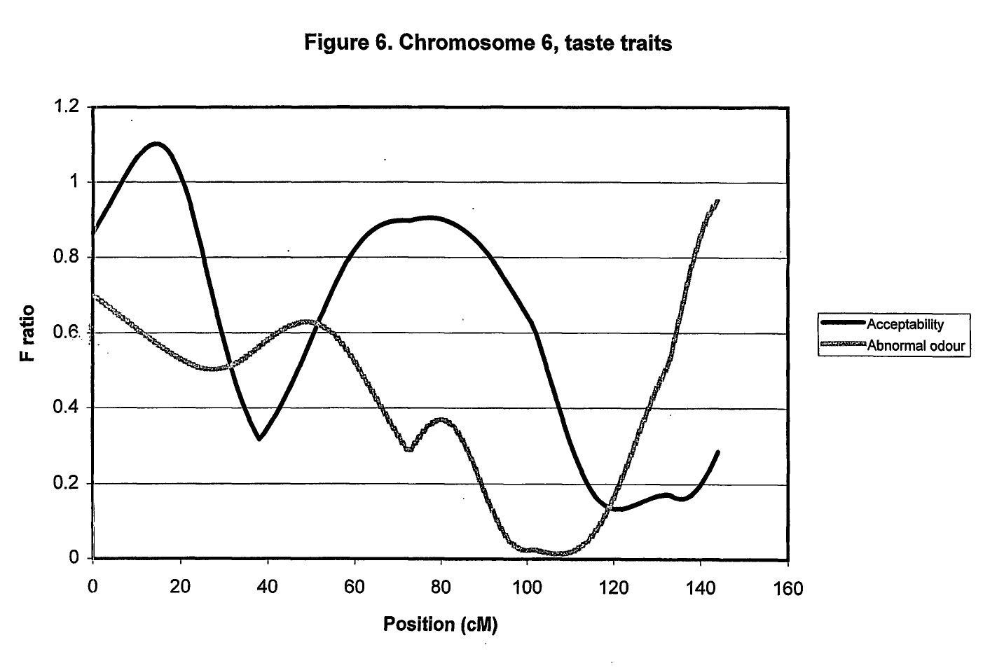

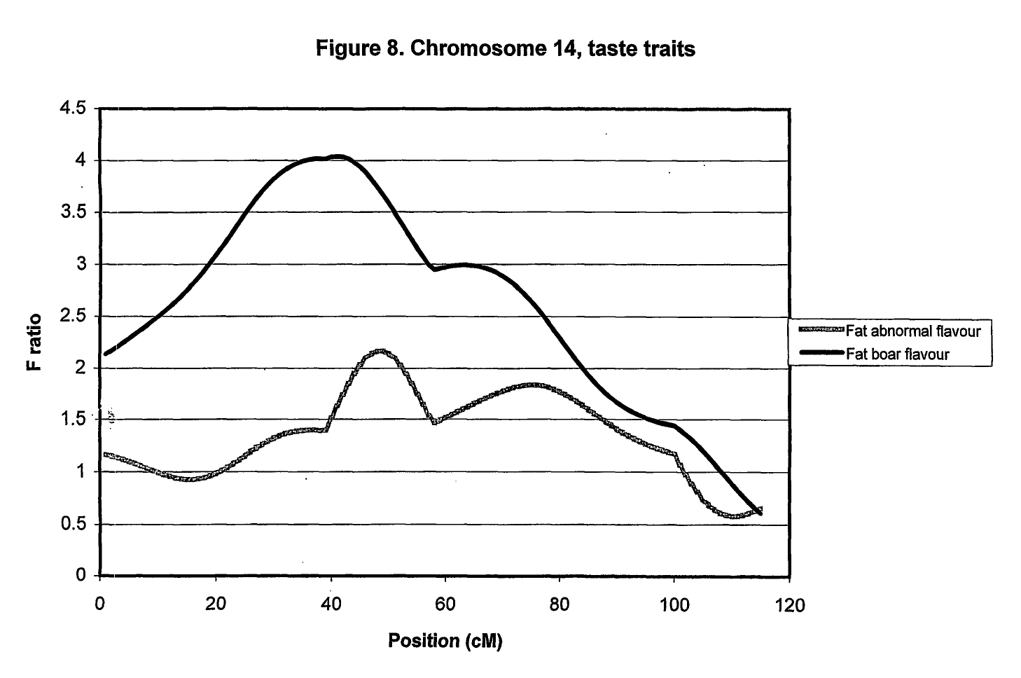

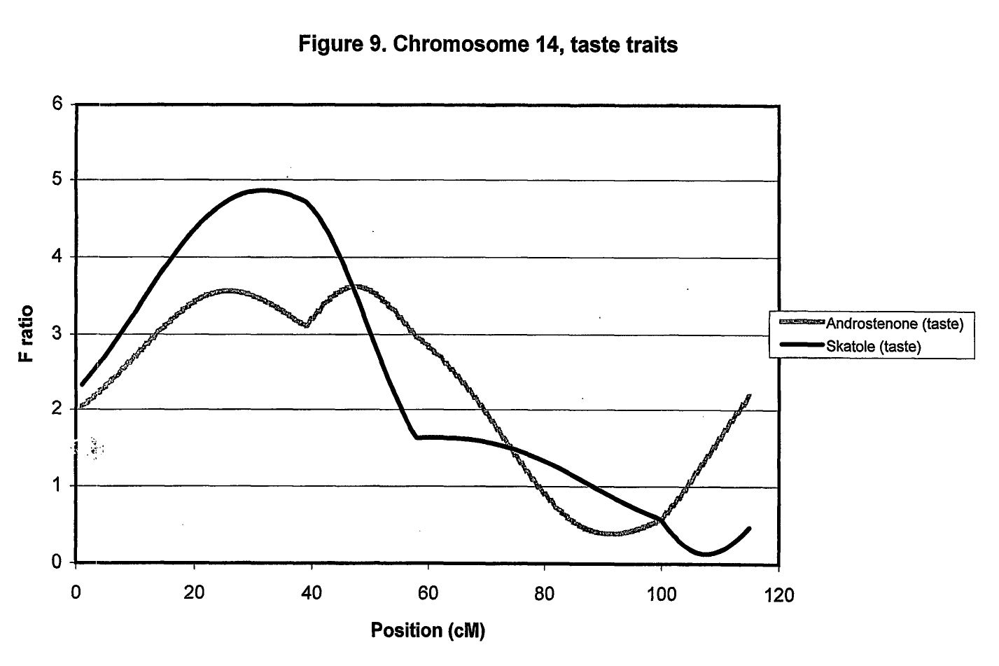

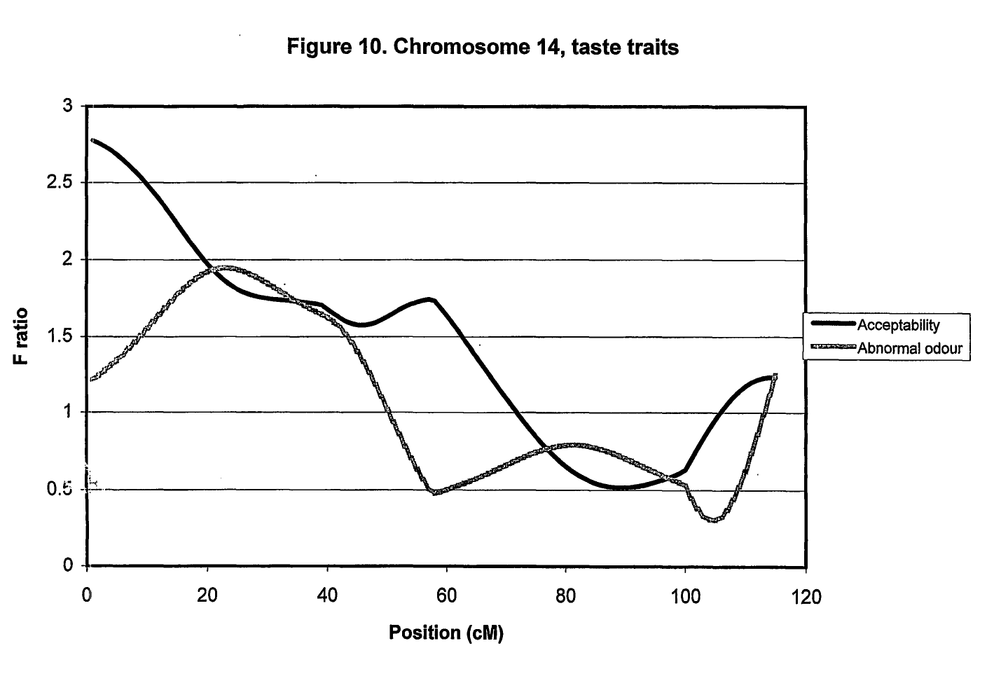

b) genotyping at least a sample of said population for pre-determined markers that map within or close to the QTL for boar taint traits defined herein (preferably on chromosomes 6 and 14, for example the specific markers referred to above or other markers located on either of chromosomes 6 and 14 where a high F ratio is indicated in any of FIGS. 1 to 10);

c) measuring boar taint traits for at least a sample of said population;

d) correlating the presence of allelic variants of said markers with said traits;

e) obtaining a DNA sample from a test pig;

f) analysing the sample to determine the allelic variant(s) present at a said genetic marker; and

g) using said marker results to select for animals of the preferred genotype.

Steps a) and d) of the method described above are concerned with identifying markers which map within or close to the QTL for boar taint traits or with confirmation that the particular markers referred to are also relevant for the test population. Preferably the markers are derived from SW782, SW1057, S0121, SW322, SW857, SW295, S0007 or SW1557.

Other markers that map within or close to the QTL described herein can also be used. Particular mention may be made of any marker located within positions 40 to 120 of chromosome 6, or within positions 10 to 70 of chromosome 14. As can be seen in FIGS. 1 to 10 certain areas of chromosomes 6 to 14 correlate to high F ratios for specific traits connected to boar taint and markers in these regions may be of particular interest.

Optionally, a selection of markers that each allow the allelic variation at different QTL associated with boar taint to be predicted may be used in combination to achieve a more accurate prediction of boar taint predisposition. The present invention thus provides a kit comprising at least two such markers, preferably selected from the specific markers recited above.

The animals shown to have marker genotypes or predicted QTL genotypes indicative of a desirable boar taint predisposition (for example boars identified to have reduced boar taint), or the close relatives of such animals, can be used as breeding stock or for meat production.

Although the genetic markers used in this study are microsatellites the assay is not limited to the use of any particular technology or type of genetic marker. Any method for detecting DNA variation at specific chromosomal locations can be used to develop genetic markers that could be used for monitoring the inheritance of particular chromosomal segments or loci. It is clear to those skilled in the art that genetic markers, which map close to or within the QTL for boar taint traits defined herein, could be used in the assay for predicting an individual's predisposition to boar taint traits independent of the technology used to develop or genotype the marker. Thus, the assay is not limited to any particular type of genetic marker or genotyping technology, current or as yet undeveloped. Other genetic marker types and technologies include, but are not limited to, restriction fragment length polymorphisms (RFLPs), single strand conformational polymorphisms (SSCP), double strand conformational polymorphisms, single nucleotide polymorphisms (SNPs), AFLP™ (amplified fragment length polymorphisms, DNA chips, variable number of tandem repeats (VNTRs, minisatellites), random amplified polymorphic DNA (RAPDs), heteroduplex analyses, and allele-specific oligonucleotides (ASOs). Some DNA variation can be detected by assaying the variation in RNA transcripts or proteins. Thus, genetic marker technology for the purposes of the assay is not limited to direct measures of DNA variation. Examples of markers that map to the boar taint QTL on chromosome 6 and 14 include, but are not limited to, (marker type and chromosome are shown in parenthesis) UBC (RFLP, SSC14); ACTA1 (PCR-RFLP, SSC14); S0063 (microsatellite, SSC14); GPI (RFLP, VNTR, protein variants, SSC6); PGD (SSCP, protein variants); TTR (SSCP, PCR-RFLP, SSC6); S0299 (microsatellite, SSC6). Details of genetic marker technology can be accessed in primary research publications, review articles, textbooks and laboratory manuals. In the assay of the present invention, the genomic DNA will be detected from a sample of porcine origin but the exact tissue forming the sample is not critical as long as it contains genomic DNA. Examples include body fluids such as blood, sperm, ascites and urine; tissue cells such as liver tissue, muscle, skin, hair follicles, fat and testicular tissue. The genomic DNA to be analysed can be prepared by extracting and purifying the DNA from such samples.

The method may be conducted in vitro or in vivo using a sample from a living animal or post mortem following the death of the animal being tested. If the assay is conducted post mortem, the information obtained may be of use for the siblings, parents or other close relatives of the animal.

The QTL for boar taint traits disclosed herein will allow the isolation and characterisation of the trait-genes themselves, since the positioning of the QTL enables a search for linkage to the genes responsible for the trait. Once these trait genes are located the option to manipulate the trait genes by transgenesis or to develop a further assay arises and forms part of the present invention.

The present invention will now be described in more detail by reference to the following, non-limiting, example and figures in which:

FIG. 1 and FIGS. 3 to 6 are graphs plotting the F value against position (cM) on chromosome 6 for different boar taint related traits.

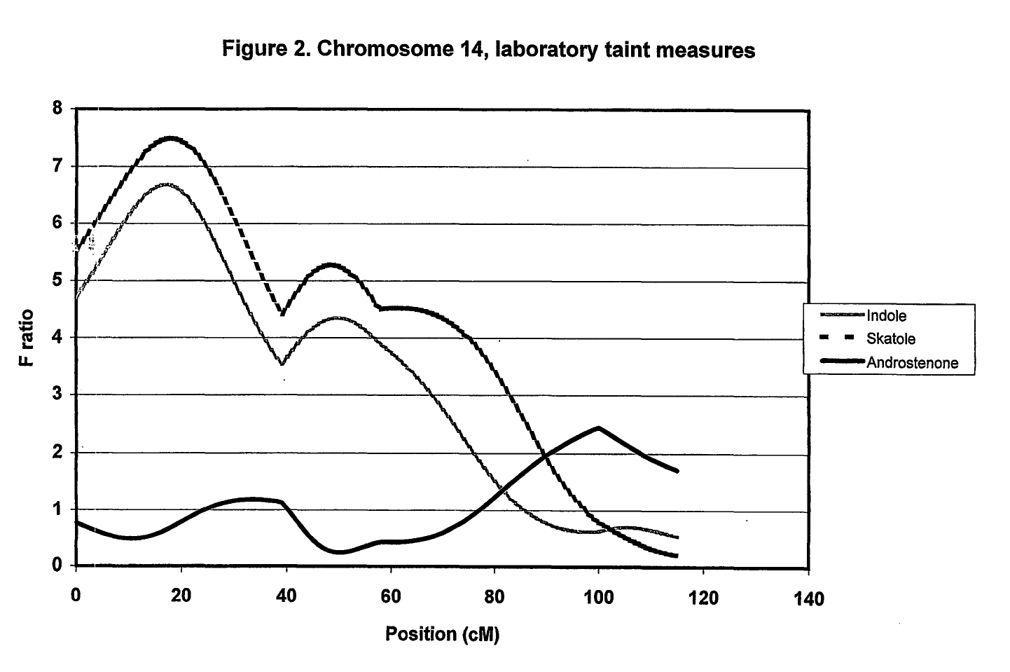

FIG. 2 and FIGS. 7 to 10 are graphs plotting the F value against position (cM) on chromosome 14 for different boar taint related traits.

FIG. 11 depicts a three-generation pig pedigree produced by crossing divergent purebred lines of pigs to produce F₁ and F₂ generations. We focus on one small part of a single chromosome that carries a genetic marker with alternative alleles 1 and 2. The animals can be genotyped for this marker and the inheritance of alternative alleles can be followed through the pedigree. In the F₂ animals, both the marker and genes controlling the size differences between the breeds segregate. The marker acts as a signpost to show from which breed linked sections of chromosome are inherited. In this example the size of F₂ animals is associated with the marker genotype (animals with the 11 genotype are large, those with 22 are small). Hence a gene or genes for size is found in the region of chromosome inherited with the marker.

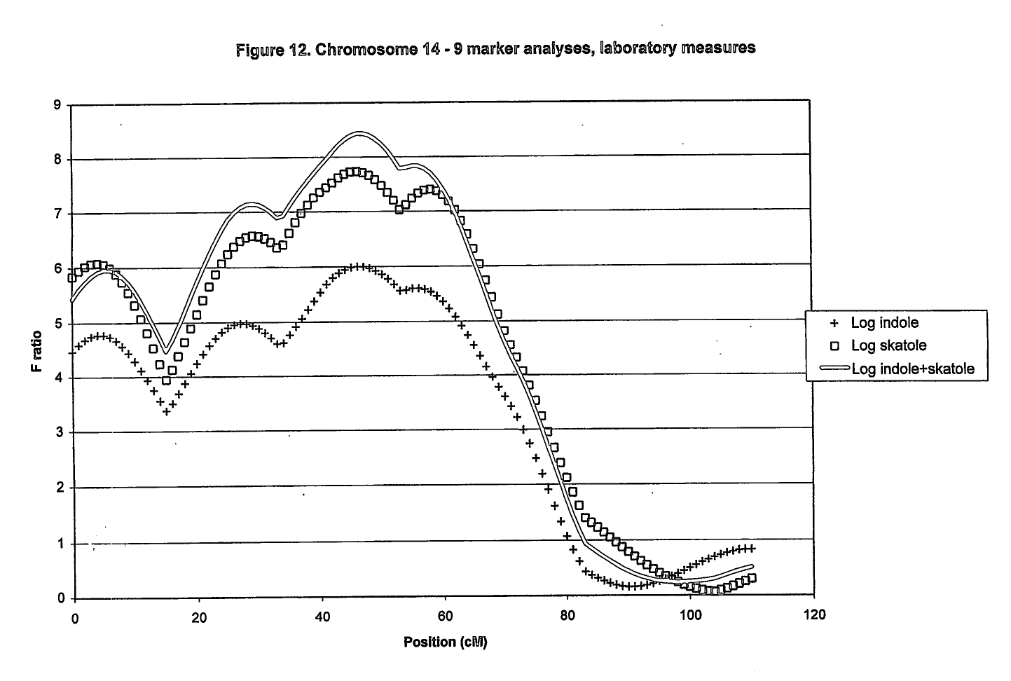

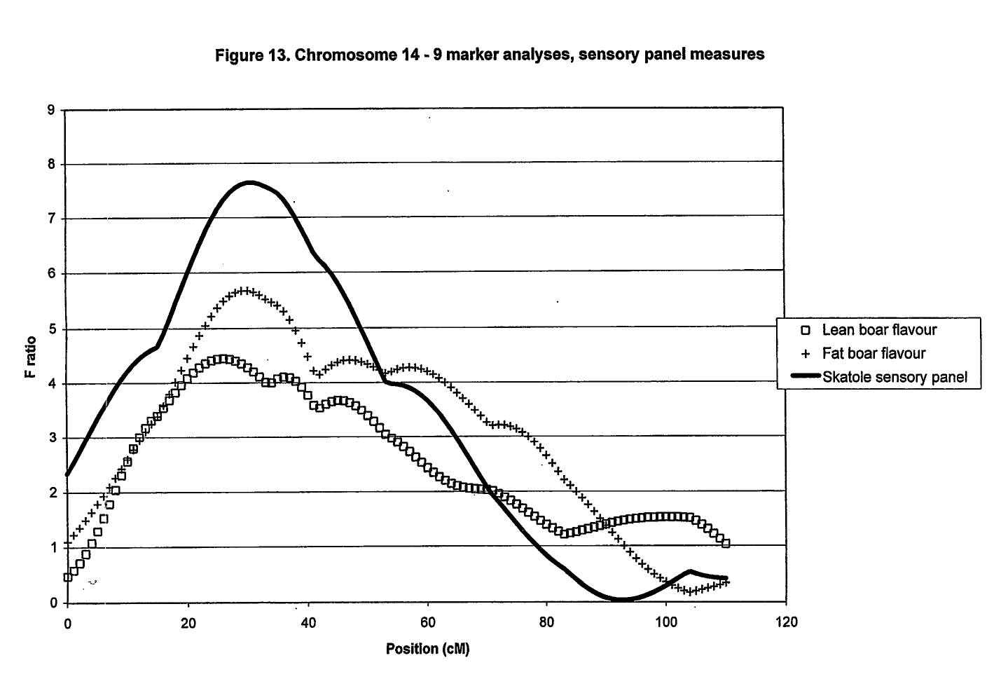

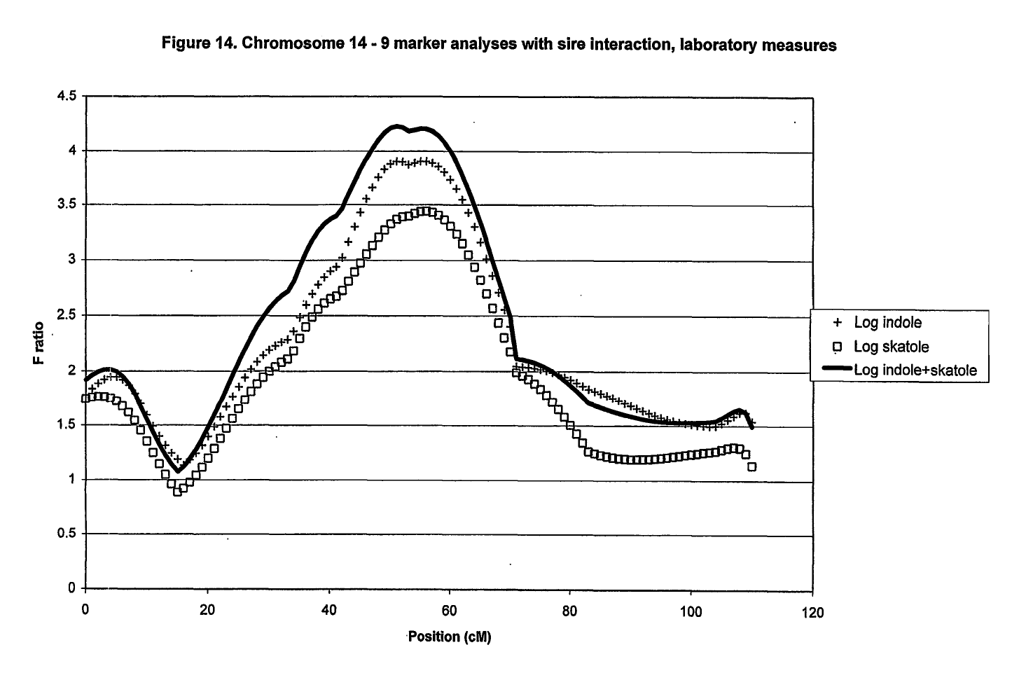

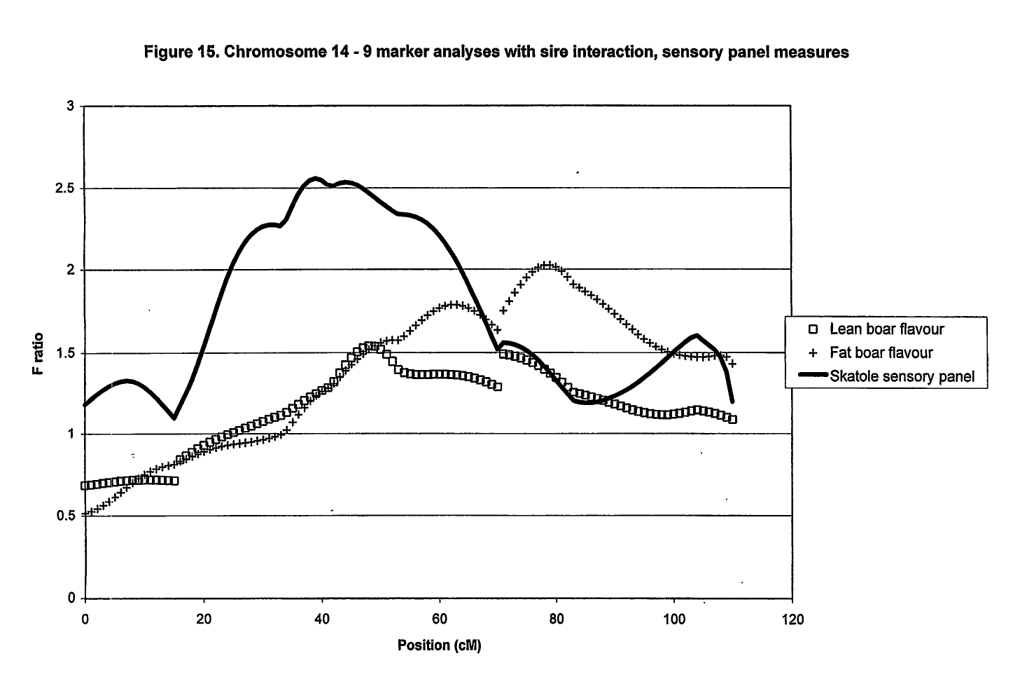

FIGS. 12 to 15 show graphs plotting the F value against position (cM) on chromosome 14 for boar taint related traits established through an alternative analysis.

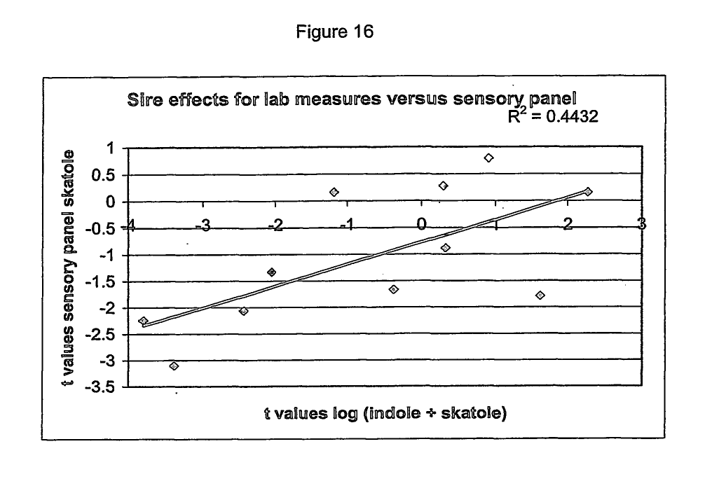

FIG. 16 shows a graph depicting the association of within sire QTL estimates for laboratory taint measures with those assessed by the taste panel.

### EXAMPLE 1

QTL mapping pedigrees were established in the form of three-generation families in which grandparents from genetically divergent breeds were crossed to produce the parental (F₁) generation which were subsequently intercrossed. The founder grandparental breeds were the Chinese Meishan and the European Large White (Yorkshire). 308 F₂ animals were produced in these Large White/Meishan pedigrees on the Roslin Institute's farm at Mountmarle, Midlothian, Scotland.

Blood samples were taken by venepuncture from most grandparental, F₁ parental and F₂ pigs. DNA was prepared from blood samples.

In the early part of the trial animals were penned in like-sex groups of 4 and fed ad libitum during the growing period. Hunday electronic feeders and weight crates were introduced for half of the second batch and all of the third batch of animals. Animals were penned in groups of 12-13 and fed ad libitum using this equipment. A comparison in the second batch showed no major differences in growth between animals penned in groups of 4 and those in larger groups with electronic feeders.

The animals were transported to the University of Bristol for slaughter at around 85 kg in weight. Phenotypic markers or component traits indicative of boar taint were analysed.

Tissue samples were taken from all F₂ animals and stored at −70° C. as a source for the preparation of DNA. DNA was prepared from frozen tissue (spleen) samples.

The phenotype markers were:

i) taste panel assessment of abnormal odour;

ii) taste panel assessment of boar flavour in lean meat;

iii) taste panel assessment of abnormal flavour in lean meat;

iv) taste panel assessment of boar flavour in fat;

v) taste panel assessment of abnormal flavour in fat;

vi) taste panel assessment of skatole;

vii) taste panel assessment of androstenone;

viii) taste panel assessment of overall acceptability.

ix) laboratory measure of indole;

x) laboratory measure of skatole;

xi) laboratory measure of androstenone;

Analysis of the phenotype markers at the University of Bristol was conducted by taste panels for items ix, x and xi using chemical analysis as described by Annor-Frempong et al., Meat Science 47:49-61, 1997; and de Brabander et al., “Boar Taint in Belgian pigs in relation to the androstenone content”, Proc. 31st Europ. Meet. Res. Works, Vama, 778-781, 1985. The remaining phenotype markers (i-viii) were measured by the trained taste panel at the Meat and Livestock Commission. Two samples of meat for each animal were assessed in separate sessions by a trained sensory panel. Over the three years of data collection, there was a total of 117 sessions, and 59 panellists were involved at some stage of the procedure, with 22 panellists appearing in all three years. At each panel session, meat samples from six animals were weighed raw, cooked, then weighed again to determine cooking loss. Each of five to seven panellists at that session was then given a separate sample of lean and fat from each of the six animals. Each panellist gave each animal a score for each of thirteen attributes, on a scale of 1-24 (the higher the better) by marking a prepared form. The lean sample was assessed by mouth for juiciness, tenderness, pork flavour, abnormal flavour and boar flavour. The fat sample was assessed by mouth for pork flavour, abnormal flavour and boar flavour and by nose for pork odour, abnormal odour, androstenone and skatole. Finally, a score was given for overall acceptability.

Each session and panellist involved in the trial had a unique number. The scores awarded by the panellists were analysed using the restricted maximum likelihood in a model fitting session number, panellist and individual animal number. Fitted values for each attribute for each individual were saved from these analyses and stored on a database for use in the QTL analyses.

DNA and tissue samples were shipped to Perkin-Elmer Agen (PE-Agen) for genotyping. Genotyping was performed using fluorescently labelled primers on ABI semi-automated DNA sequencers. The size of the labelled PCR products as resolved on ABI semi-automated DNA sequencers was estimated using ABI proprietary software (Genescan™ and Genotyper™). Genotyping results were returned to the Roslin Institute on CD-ROM. The results were loaded into the project database (resSpecies-pig http://www.ri.bbsrc.ac.uk/bioinformatics/databases).

Details of the pedigree structure, dates of birth, sex and growth and feed intake were loaded into resSpecies from the farm database.

The collated data on traits and marker genotypes were analysed to scan the genome for the presence of QTL influencing the traits of interest.

The animals were genotyped for the genetic markers listed in Table 1. The markers were chosen to provide a reasonable spread over the whole of the genome.

TABLE 1
Markers used for genome scan

| Marker | Chromosome | Position (cM) |
|---|---|---|
| SW1515 | 1 | 0.0 |
| CGA | 1 | 41.9 |
| S0082 | 1 | 69.8 |
| S0155 | 1 | 77.3 |
| SW1828 | 1 | 105.7 |
| SW373 | 1 | 109.1 |
| SW1301 | 1 | 131.2 |
| SW2443 | 2 | 0.0 |
| SW256 | 2 | 20.1 |
| SW240 | 2 | 49.2 |
| S0226 | 2 | 73.7 |
| S0378 | 2 | 92.3 |
| S0036 | 2 | 130.9 |
| SW72 | 3 | 0.0 |
| SW2527 | 3 | 20.8 |
| SW902 | 3 | 39.2 |
| S0167 | 3 | 70.1 |
| S0002 | 3 | 92.0 |
| SW590 | 3 | 116.3 |
| S0227 | 4 | 0.0 |
| S0301 | 4 | 23.9 |
| S0001 | 4 | 43.4 |
| S0217 | 4 | 61.5 |
| S0073 | 4 | 67.8 |
| SW445 | 4 | 99.4 |
| S0097 | 4 | 117.1 |
| DAGK | 5 | 0.0 |
| S0005 | 5 | 15.2 |
| IGF1 | 5 | 40.8 |
| SW1954 | 5 | 54.2 |
| SW967 | 5 | 77.3 |
| SW2535 | 6 | 0.0 |
| SW1057 | 6 | 38.1 |
| SW782 | 6 | 72.5 |
| S0121 | 6 | 101.5 |
| SW322 | 6 | 132.6 |
| SW2419 | 6 | 144.4 |
| S0025 | 7 | 0.0 |
| SW2155 | 7 | 34.9 |
| TNFB | 7 | 59.6 |
| S0066 | 7 | 76.8 |
| SW632 | 7 | 98.7 |
| S0101 | 7 | 124.0 |
| SW764 | 7 | 145.4 |
| SW2611 | 8 | 0.0 |
| S0017 | 8 | 72.0 |
| S0225 | 8 | 87.6 |
| SW61 | 8 | 111.2 |
| S0178 | 8 | 144.9 |
| SW983 | 9 | 0.0 |
| SW911 | 9 | 34.7 |
| SW1677 | 9 | 69.3 |
| SW2093 | 9 | 92.0 |
| SW1651 | 9 | 166.0 |
| SW830 | 10 | 0.0 |
| SW443 | 10 | 31.7 |
| SW497 | 10 | 54.0 |
| SW1041 | 10 | 70.3 |
| SW951 | 10 | 98.8 |
| SWR67 | 10 | 129.9 |
| S0385 | 11 | 0.0 |
| SW1632 | 11 | 18.8 |
| S0071 | 11 | 41.2 |
| S0230 | 11 | 51.6 |
| SW703 | 11 | 70.0 |
| S0143 | 12 | 0.0 |
| SW957 | 12 | 19.3 |
| S0090 | 12 | 49.9 |
| SW1378 | 13 | 0.0 |
| S0076 | 13 | 14.9 |
| S0068 | 13 | 53.3 |
| SW398 | 13 | 71.7 |
| SW1056 | 13 | 93.3 |
| S0215 | 13 | 113.3 |
| SW857 | 14 | 0.0 |
| SW2496 | 14 | 15.1 |
| SW295 | 14 | 41.5 |
| S0007 | 14 | 53.2 |
| SW761 | 14 | 70.6 |
| SW1557 | 14 | 83.0 |
| SW2515 | 14 | 103.8 |
| SWC27 | 14 | 110.9 |
| S0355 | 15 | 0.0 |
| S0148 | 15 | 14.5 |
| SW964 | 15 | 26.7 |
| SW936 | 15 | 54.3 |
| SW1119 | 15 | 84.4 |
| S0111 | 16 | 0.0 |
| S0006 | 16 | 51.5 |
| S0026 | 16 | 89.5 |
| SW1897 | 16 | 110.0 |
| SW24 | 17 | 0.0 |
| SW1920 | 17 | 31.1 |
| S0332 | 17 | 63.4 |
| SW2540 | 18 | 0.0 |
| SW1984 | 18 | 28.8 |
| SW1682 | 18 | 41.0 |
| SW949 | X | 0.0 |
| SW2534 | X | 57.8 |
| SW2456 | X | 70.1 |
| SW1943 | X | 82.5 |
| S0218 | X | 94.2 |

Linkage maps of each pig chromosome were developed using Cri-Map version 2.4 (Green, P., Falls, K. and Crooks, S. (1990), Documentation for Cri-Map version 2.4. St. Louis, Washington University School of Medicine). The linkage map positions for the markers on chromosomes 6 and 14 are presented in Table 1.

The trait data and linkage maps were analysed by the least squares approach as described by Haley et al., Genetics, 136:1195-1207, 1994. Due to the non-normality of the laboratory measured traits indole, skatole and androstenone, data for these traits were log-transformed prior to analysis. All chromosomes were tested in this way (using appropriate markers for the chromosome under test), but the most significant correlation was found for boar taint in the markers for chromosomes 6 and 14.

Other more minor effects for the laboratory measured traits are given below in Table 2 (two sexes analysed separately and with log transformed data):

TABLE 2

| Chromosome | Trait |
|---|---|
| 2 | Skatole |
| 4 | Skatole, androstenone |
| 7 | Androstenone |
| 8 | Androstenone, indole |
| 9 | Androstenone |
| 11 | Skatole, androstenone, indole |
| 12 | Skatole |
| 13 | Androstenone, indole |
| 16 | Androstenone |
| 17 | Androstenone |
| X | Skatole, androstenone, indole |

Brief details of the markers found to map to QTL for boar taint are given below:

SW782: Rohrer et al., “A microsatellite linkage map of the porcine genome”, Genetics 136:231-45, 1994.

Method: Microsatellite

| | |
|---|---|
| Forward Primer: | TCTTCACATATGAGCACCAACC |
| Reverse Primer: | CGGAACAAGAGGAAGTGAGTG |

PCR Conditions:

Anneal temp 60.000° C.

Mg²⁺conc 1.500 mM

dNTPs-conc 30.00 μM

PCR-Annotation 12.5 ng DNA template, 5 pmol each primer, 0.45 units Taq polymerase. For further details of allele size range and heterozygosity see http://sol.marc.usda.gov.

Gel Details:

Matrix: polyacrylamide Concentration: 7.000 g/100 ml

S0121 (6 q3.1-q3.5): Robic et al., “Porcine linkage and cytogenetic maps integrated by regional mapping of 100 microsatellites on somatic cell hybrid panel”, Mammalian Genome 7:438:445, 1996.

EMBL Accession No L30152

Method: Microsatellite

| | |
|---|---|
| Forward Primer: | TTGTACAATCCCAGTGGAATCC |
| Reverse Primer: | AATAGGGCATGAGGGTGTTTGA |

PCR Conditions:

Anneal temp 55.000° C.

Mg²⁺conc 2.000 mM

dNTPs-conc 200.000 μM

Cycle profile 6 min at 94° C., 30×1 min at 55° C.; 1 min at 72° C.; 1 min at 94° C., followed by a final extension of 7 min at 72° C.

Gel Details:

Matrix polyacrylamide

Concentration 6.000 g/100 ml

Additives 7M urea

SW322 (6 q3.1-q3.5): Rohrer at al., 1994, supra; Robic et al., 1996, supra.

Method: Microsatellite

| | |
|---|---|
| Forward Primer: | CATTCAACCTGGAATCTGGG |
| Reverse Primer: | TCCCTGGAAAGGCTACACC |

PCR Conditions

Anneal temp 62.000° C.

Mg++conc 1.500 mM

dNTPs-conc 30.000 μM

PCR-Annotation 12.5 ng DNA template, 5 pmol each primer, 0.45 units Taq polymerase. For further deatils of allele size range and heterozygosity see http://sol.marc.usda.gov

Gel Details

Matrix: polyacrylamide

Concentration 7.000 g/100 ml

SW857 (14 q2.1-q2.2): Lopez-Corrales et al., “Cytogenic assignment of 53 microsatellites from the USDA-MARC porcine genetic map”, Cytogenetics and Cell Genetics 84:140-144, 1999.

Method: Microsatellite

| | |
|---|---|
| Forward Primer: | TGAGAGGTCAGTTACAGAAGACC |
| Reverse Primer: | GATCCTCCTCCAAATCCCAT |

PCR Conditions:

Anneal temp 58.000° C.

Mg²⁺conc 1.500 mM

dNTPs-conc 30.000 μM

PCR-Annotation 12.5 ng DNA template, 5 pmol each primer, 0.45 units Taq polymerase. For further details of allele size range and heterozygosity see http://sol.marc.usda.gov.

Gel Details:

Matrix polyacrylamide

Concentration 7.000 g/100 ml

SW295 (14 q2.2-q2.4): Robic et al., 1996, supra.

Method: Microsatellite

| | |
|---|---|
| Forward Primer: | ACCTGCCAGAGTTGTGGC |
| Reverse Primer: | AAGAGTTTCATTTCTCCCATCC |

PCR Conditions:

Anneal temp 62.000° C.

Mg²⁺conc 1.500 mM

dNTPs-conc 30.000 μM

PCR-Annotation 12.5 ng DNA template, 5 pmol each primer, 0.45 units Taq polymerase. For further details of allele size range and heterozygosity see http://sol.marc.usda.gov.

Gel Details:

Matrix polyacrylamide

Concentration 7.000 g/100 ml

S0007 (14) Fredholm et al., “Characterization of 24 porcine (dA-dC)n-(dT-dG)n microsatellites: genotyping of unrelated animals from four breeds and linkage studies”, Mammalian Genome 4:187-92, 1993.

EMBL Accession No M97234

Method: Microsatellite

| | |
|---|---|
| Forward Primer: | TTACTTCTTTGGATCATGTC |
| Reverse Primer: | GTCCCTCCTCATAATTTCTG |

PCR Conditions:

Anneal temp 56.000° C.

Mg²⁺conc 1.500 mM

Salt-conc 50.000 mM

dNTPs-conc 200.000 μM

Cycle profile 1×94° C., 3 min; 56° C., 1 min; 72° C., 30 sec; then 30×94° C., 30 sec; 56° C., 1 min; 72° C., 5 min.

PCR-Annotation Hybaid thermal cycler

Gel Details:

Matrix polyacrylamide

Concentration 6.000 g/100 ml

Additives denaturing gel

SW1557 (14) Alexander et al., “Cloning and characterization of 414 polymorphic porcine microsatellites”, Animal Genetics 27:137-148, 1996.

Method: Microsatellite

| | |
|---|---|
| Forward Primer: | TGCTCTAATCTACCCGGGTC |
| Reverse Primer: | CCACCCCACTCCCTTCTG |

PCR Conditions:

Anneal temp 58.000° C.

Mg²⁺conc 1.500 mM

dNTPs-conc 30.000 μM

Cycle profile 92° C., 2 min; 30×94° C., 30 sec, anneal temp 30 sec, 72° C. 30 sec; 1×72° C., 5 min.

PCR-Annotation 12.5 ng DNA template, 5 pmol each primer, 0.45 units Taq polymerase. For further details of allele size range and heterozygosity see USDA-MARC database—http://sol.marc.usda.gov.

Gel Details:

Matrix polyacrylamide

Concentration 7.000 g/100 ml

SW2496 (14 q2.1-q2.2): Lopez-Corrales et al. “Cytogenetic assignment of 53 microsatellites from the USDA-MARC porcine genetic map”, Cytogenetics and Cell Genetics 84:140-144, 1999.

Method: Microsatellite

| | |
|---|---|
| Forward Primer: | TGAGAGGTCAGTTACAGAAGACC |
| Reverse Primer: | GATCCTCCTCCAAATCCCAT |

PCR Conditions

Anneal temp 58.000° C.

Mg++conc 1.500 mM

dNTPs-conc 30.000 μM

PCR-Annotation 12.5 ng DNA template, 5 pmol each primer, 0.45 units Taq polymerase. For further details of allele size range and heterozygosity see http://sol.marc.usda.gov

Gel Details

Matrix: polyacrylamide

Concentration 7.000 g/100 ml

SW210: Rohrer et al. “A microsatellite linkage map of the porcine genome.” Genetics 136:231-45, 1994.

Method: Microsatellite

| | |
|---|---|
| Forward Primer: | TCATCACCATCATACCAAGATG |
| Reverse Primer: | AATTCTGCCAAGAAGAGAGCC |

PCR Conditions

Anneal temp 60.000° C.

Mg++conc 1.500 mM

dNTPs-conc 30.000 μM

PCR-Annotation: 12.5 ng DNA template, 5 pmol each primer, 0.45 units Taq polymerase. For further details of allele size range and heterozygosity see http://sol.marc.usda.gov

Gel Details

Matrix: polyacrylamide

Concentration. 7.000 g/100 ml

SW761 Rohrer et al. “A microsatellite linkage map of the porcine genome.” Genetics 136:231-45, 1994.

Method: Microsatellite

| | |
|---|---|
| Forward Primer: | CTTTGCTCCCCATTAAGCTG |
| Reverse Primer: | TCTAGCAAATGTCTGAGATGCC |

PCR Conditions

Anneal temp 60.000° C.

Mg++conc 1.500 mM

dNTPs-conc 30.000 μM

PCR-Annotation 12.5 ng DNA template, 5 pmol each primer, 0.45 units Taq polymerase. For further details of allele size range and heterozygosity see http://sol.marc.usda.gov

Gel Details

Matrix: polyacrylamide

Concentration 7.000 g/100 ml

SW2515 (14 q 2.9) Alexander et al. “Physical assignments of 68 porcine cosmids and lambda clones containing microsatellites.” Mammalian Genome 7:368-372, 1996.

Method: Microsatellite

| | |
|---|---|
| Forward Primer: | CCATCTCATCCAGAAACATCC |
| Reverse Primer: | AGGATGCTGAGGTGTTAGGC |

PCR Conditions

Anneal temp 60.000° C.

Mg++conc 1.500 mM

dNTPs-conc 30.000 μM

PCR-Annotation 12.5 ng DNA template, 5 pmol each primer, 0.45 units Taq polymerase. For further details of allele size range and heterozygosity see http://sol.marc.usda.gov

Gel Details

Matrix: polyacrylamide

Concentration 7.000 g/100 ml

SWC27 (14 q2.8-q2.9) Alexander et al. “Physical assignments of 68 porcine cosmids and lambda clones containing microsatellites.” Mammalian Genome 7:368-372, 1996.

Method: Microsatellite

| | |
|---|---|
| Forward Primer: | CATTCAACCTGGAATCTGGG |
| Reverse Primer: | TCCCTGGAAAGGCTACACC |

PCR Conditions

Anneal temp 58.000° C.

Mg++conc 1.500 mM

dNTPs-conc 30.000 μM

PCR-Annotation 12.5 ng DNA template, 5 pmol each primer, 0.45 units Taq polymerase. For further deatils of allele size range and heterozygosity see http://sol.marc.usda.gov

Gel Details

Matrix: polyacrylamide

Concentration 7.000 g/100 ml

### QTL Analyses

All QTL analyses were performed by least squares. The assumption underlying these analyses is that QTL of major (i.e. detectable) effects were fixed for alternative alleles in the Meishan and Large White breeds that went into the study.

Several alternative models were used in the QTL analyses. The basic models included fixed effects and any key covariates. Sex was always included as was either year or slaughter data as a fixed effect. For traits where QTL effects may differ between sexes a model including a QTL×sex interaction (estimating a separate QTL effect for both sexes) was used in addition to the basic model.

### Results

The significant results for log transformed data and analysis allowing for differences between the sexes are set out in Table 3.

From Table 3 it can be seen that when analysis of androstenone, indole and skatole was performed on the basis of the sex of the animal, it was found that no QTL effect was present in female pigs, as expected (estimates of additive and dominance effects in females were not significantly different from zero), but significant effects were found in males.

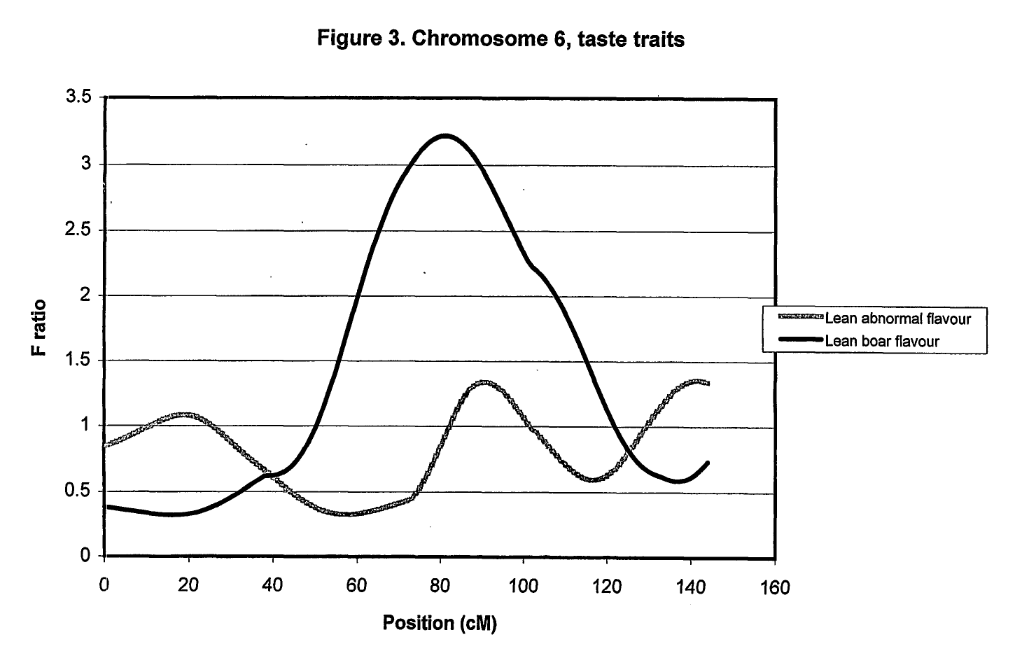

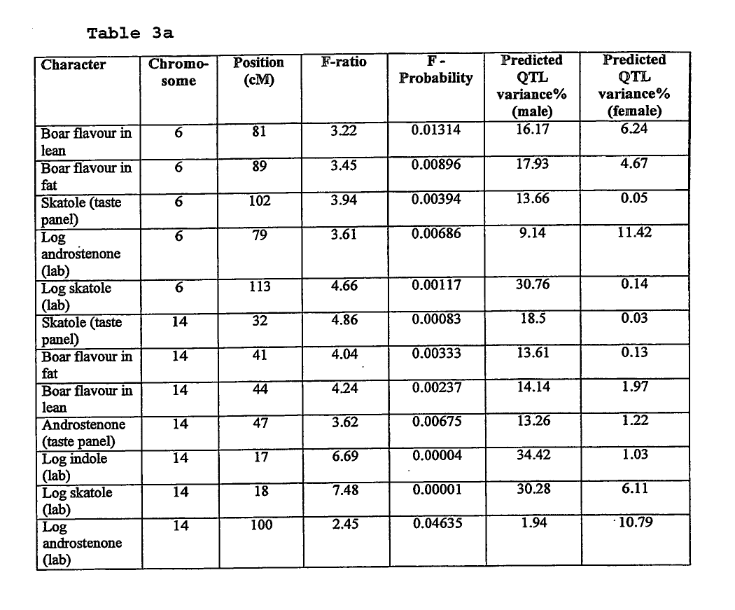

The results of the analysis for chromosome 6 are summarised in FIG. 1 for laboratory measurements of taint associated compounds and in FIGS. 3 to 6 for traits recorded by the taste panel. These Figures show that high F values peak on chromosome 6 at positions 40 to 120.

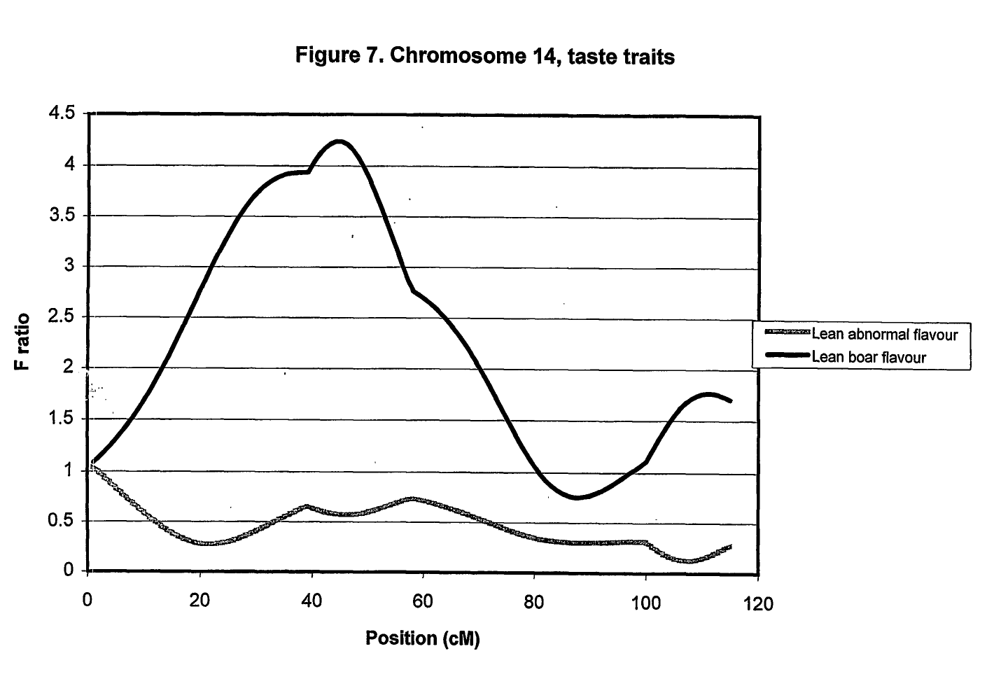

The results of the analysis for chromosome 14 are summarised in FIG. 2 for laboratory measurements of taint associated compounds and in FIGS. 7 to 10 for traits recorded by the taste panel. These Figures show that high F values peak on chromosome 14 at positions 10 to 70.

Unexpectedly, and contra-indicated by the literature, our results indicate an association between skatole and androstenone and this ability to use both markers together to measure boar taint predisposition will significantly enhance the accuracy of the assay.

### Further QTL Analysis

In view of the findings and conclusions drawn from the QTL analysis as set out above, further analysis was carried out, this analysis looking specifically at log transformed laboratory measures of indole and skatole, as well as the most important measures of taint as assessed by the sensory panel.

It should be noted that these analyses, unlike the analysis previously shown, was carried out using data from males only. The basis for this was that previous analysis had included both sexes, but allowed the QTL effect to differ between sexes. There was however no evidence in the earlier analysis to show any effect of detected QTL in females, hence females are excluded from the present analysis.

This analysis further served to establish a new trait by summing the laboratory measures of indole and skatole and include a measure of the log (indole+skatole) in the analysis, wherein these measurements were only analysed separately in the previous analysis.

An additional analysis was included that looked at whether QTL effects differed according to F1 sire (sire interaction). Previous analyses made assumption that any QTL was fixed for alternative alleles in the two breeds (Meishan and Large White) crossed. This means that all F1 parents should be the same for any QTL and all F2 litters should be segregating in a similar manner. This new analysis allows F1 sires to differ from one another, as they would if a QTL was segregating within either or both of the two breeds (Meishan and Large White).

### Results

Data were available on 180 F2 males, progeny of 11 F1 sires.

Analyses of log transformed data on laboratory measures (skatole, indole and skatole+indole) gave less clear and lower peak at 46 cM (between SW210 and S0007). These peaks were significant at the suggestive level (F=6.0 to 8.3).

Sensory panel data provided evidence for QTL particularly for ‘skatole’ (F=7.65 at 31 cM) and fat boar flavour (F=5.68 at 30 cM).

Detailed estimates from these analyses are shown in table 4.

Analyses of (log) laboratory indole, skatole and indole+skatole measures including a sire interaction increased the significance level to genome wide significance and the interaction with sire was significant. The estimated QTL position was 51-56 cM, close to S0007. Test statistics and estimated position of the QTL are given in table 5 below.

TABLE 5

| Character | Chr. | Position (cM) | F-ratio | F-probability |
|---|---|---|---|---|
| Boar flavour | 14 | 48 | 1.54 | 0.12213 |
| in lean | | | | |
| Boar flavour | 14 | 79 | 2.02 | 0.02971 |
| in fat | | | | |
| Skatole | 14 | 39 | 2.56 | 0.00521 |
| (sensory | | | | |
| panel) | | | | |
| Log skatole | 14 | 56 | 3.44 | 0.00026 |
| (lab) | | | | |
| Log indole | 14 | 56 | 3.91 | 0.00005 |
| (lab) | | | | |
| Log | 14 | 51 | 4.23 | 0.00002 |
| indole + skatole (lab) | | | | |

Some sires showed a positive QTL effect and others a negative QTL effect, although as in the foregoing analyses, the overall effect was negative (indicating that an average Large White alleles reduce levels).

Results were less clear cut for sire interaction analysis of sensory panel assessment of skatole. To look at the association of within sire QTL estimates for laboratory taint measures with those assessed by the taste panel, estimated the association between the within sire QTL t-values (estimated within sire QTL estimate divided by its standard error) for the two analyses of log (indole+skatole) and the sensory panel assessment of skatole. The plot of these estimates for the 11 F1 sires is shown in FIG. 16. This figure shows that across sires there are both negative and positive within sire QTL estimates for both laboratory and sensory panel taint measures and these estimates were well correlated (r=0.66) across sires.

These results confirm that the QTL must be segregating within one or both of the two breeds originally crossed as well as in the cross between them. The within sire segregation of taint measures recorded in the laboratory provides a good predictor of taint as assessed by a sensory panel. Hence the QTL may potentially be used as a predictor of taint within European populations as well as in experimental crosses.

### Chromosomal Localization of CYP2E Candidate Gene

To localise the (candidate) CYP2E (cytochrome P450, subfamily IIE (ethanol-inducible) gene on the porcine genome, two PCR tests were developed to amplify porcine CYP2E sequences from a porcine—rodent somatic cell hybrid panel of twenty-seven cell lines (Yerle, M., Echard, G., Robic, A., Mairal, A., Dubut Fontana, C., Riquet, J., Pinton, P., Milan, D., Lahbib-Mansais, Y. and Gellin, J., 1996. A somatic cell hybrid panel for pig regional gene mapping characterized by molecular cytogenetics. Cytogenetics and Cell Genetics 73: 194). The PCR reactions were optimised for temperature, magnesiium concentration and the number of cycles to specifically amplify the porcine gene only. One pair of gene-specific oligonucleotide primers (sequences CYP2E7.for and CYP2E8.rev) were designed for amplification of a fragment spanning the predicted intron between the predicted exons 7 and 8.

CYP2E7.for 5′-CATGAGATTCAGCGATTCATCG-3′
CYP2E8.rev 5′-TGCTCTGGCTTAAACTTCTCCG-3′

Both PCR reactions contained the relevant pair of gene-specific oligonucleotide primers at a concentration of 0.2 micromolar and 50 nanograms of porcine/rodent somatic cell hybrid cell line genomic DNA. Control samples included hamster genomic DNA (50 nanograms), mouse genomic DNA (50 nanograms) and porcine genomic DNA (50 nanograms). Aliquots of the PCR products were examined by agarose (1.2% w/v) gel electrophoresis. Each gel lane was scored for the presence or absence of the expected porcine-specific CYP2E gene-specific PCR product. Statistical analysis of these data was performed with a computer program available on the World Wide Web (Chevalet, C., Gouzy, J. and SanCristobal Gaudy, M., 1997. Regional assignment of genetic markers using a somatic cell hybrid panel: a WWW interactive program available for the pig genome. Computer Applications in BioScience 13: 69).

Analysis of the pattern of presence or absence of the pig CYP2E gene-specific sequences across the panel of porcine-rodent somatic cell hybrids suggested that the CYP2E gene maps to either chromosome 14 or 6 (SSC14 or 6).

TABLE 4

| | | | | | Predicted | | Male | | Male | |
|---|---|---|---|---|---|---|---|---|---|---|
| | | Position | F- | F-pro- | QTL | Trait | additive | | dominance | |
| Character | Chr. | (cM) | ratio | bability | variance | s.d. | effect | s.e. | effect | s.e. |
| Boar | 14 | 26 | 4.45 | 0.01314 | 8.50% | 1.847 | −0.578 | 0.236 | 0.704 | 0.38 |
| flavour | | | | | | | | | | |
| in lean | | | | | | | | | | |
| Boar | 14 | 30 | 5.68 | 0.00413 | 9.80% | 2.132 | −0.778 | 0.261 | 0.75 | 0.405 |
| flavour | | | | | | | | | | |
| in fat | | | | | | | | | | |
| Skatole | 14 | 31 | 7.65 | 0.00067 | 11.40% | 2.368 | −1.097 | 0.282 | 0.381 | 0.432 |
| (sensory | | | | | | | | | | |
| panel) | | | | | | | | | | |
| Log | 14 | 46 | 7.74 | 0.00062 | 11.40% | 0.471 | −0.212 | 0.059 | −0.105 | 0.089 |
| skatole | | | | | | | | | | |
| (lab) | | | | | | | | | | |
| Log | 14 | 46 | 6 | 0.00306 | 8.90% | 0.436 | −0.168 | 0.055 | −0.108 | 0.083 |
| indole | | | | | | | | | | |
| (lab) | | | | | | | | | | |
| Log | 14 | 47 | 8.43 | 0.00033 | 12.30% | 0.409 | −0.192 | 0.051 | −0.093 | 0.077 |
| indole + | | | | | | | | | | |
| skatole | | | | | | | | | | |
| (lab) | | | | | | | | | | |

1. A method for determining whether a pig is predisposed to boar taint comprising assaying for the presence of alleles conveying susceptibility to boar taint using genetic markers selected from the group SW1057, SW782, SW1057, S0121, SW322 or regions of chromosome 6 spanning therebetween.

2. A method for determining whether a pig is predisposed to boar taint comprising an assay for the presence of alleles conveying susceptibility to boar taint using genetic markers selected from the group SW857, SW2496, SW295, SW210, S0007, SW761, SW1557 or regions of chromosome 14 spanning therebetween.

3. An assay to identify pigs with a genetic predisposition that reduces the incidence of boar taint, wherein said assay comprises;
obtaining a DNA sample from a test pig,
analysing said sample to determine the allelic variants present at least one genetic marker, wherein said marker is selected from;
SW1057, SW782, SW1057, S0121, SW322 or regions of chromosome 6 spanning therebetween;
and SW857, SW2496, SW295, SW210, S0007, SW761, SW1557 or regions of chromosome 14 spanning therebetween;
and using the genotypic data from said marker(s) to select for pigs of preferred genotype.

4. A method of identifying boars which have a genetic disposition to reduce boar taint, said method comprising obtaining a DNA sample from said boar, and
assaying said boar for genotypes for at least one of the genetic markers identified in claim 3.

5. A method to identify pigs with a genetic predisposition which reduces the incidence of boar taint wherein said method comprises;
obtaining DNA samples from a population of pigs;
genotyping at least a sample of said population for pre-determined markers that map within or close to the QTL for boar taint traits on chromosomes 6 and 14, using markers referred to above or other markers located on either of chromosomes 6 and 14 at a location displaying a high F ratio;
measuring boar taint traits for at least a sample of said population;
correlating the presence of allelic variants of said markers with said traits;
obtaining a DNA sample from a test pig;
analysing the sample to determine the allelic variant(s) present at a said genetic marker; and
using said marker results to select for pigs of the preferred genotype.

6. A method of identifying boars which are genetically predisposed for reduced boar taint, comprising obtaining a DNA sample from said boar and assaying said sample for genetic variants in the CYP2E gene on chromosome 6 or 14 or in the region of the genome linked to this gene.

7. A method of detecting the predisposition to boar taint comprising the detection of genes located between the positions of the genetic markers SW1057 and SW322 on chromosome 6, variation in which can influence boar taint or its component traits.

8. A method of detecting the predisposition to boar taint comprising the detection of genes located between the position of the genetic markers SW857 and SW1557 on chromosome 14, variation in which can influence boar taint or its component traits.

9. A method of detecting the predisposition to boar taint comprising the detection of markers located between the positions of the genetic amrkers SW1057 and SW322 on chromosome 6, variation in which can influence boar taint or its component traits.

10. A method of detecting the predisposition to boar taint comprising the detection of markers located between the position of the genetic markers SW857 and SW1557 on chromosome 14, variation in which can influence boar taint or its component traits.
